(19) United States
(12) Patent Application Publication
(10) Pub. No.: US 2024/0336937 A1
(43) Pub. Date: Oct. 10, 2024

(54) METHOD FOR CELL TRANSDUCTION AND MICROFLUIDIC CHIP FOR CELL TRANSDUCTION

(71) Applicant: CTCELLS, INC., Daegu (KR)

(72) Inventor: Jung Min LEE, Daegu (KR)

(21) Appl. No.: 18/285,057

(22) PCT Filed: Mar. 28, 2022
(86) PCT No.: PCT/KR2022/004358

§ 371 Date: Apr. 29, 2024

(30) Foreign Application Priority Data
Mar. 31, 2021 (KR) 10-2021-0042401

(51) Int. Cl. C12N 15/87; C12M 3/06

(52) U.S. Cl. CPC C12N 15/87; C12M 23/16; C12N 2740/15043

(57) ABSTRACT

Provided are a cell transduction method and a microfluidic chip for cell transduction. According to the present disclosure, since traits are introduced using a microfluidic-based method, it is possible to avoid cell stress induced by strong centrifugal force and to achieve a high multiplicity of infection (MOI) even with a small volume, thereby being efficiently used for cell transformation.

### TECHNICAL FIELD

The present disclosure relates to a cell transduction method and a microfluidic chip for cell transduction.

### BACKGROUND ART

Transduction generally refers to introduction of genetic materials into cells, and has contributed greatly to molecular and recombinant innovations in biology. For transduction into higher eukaryotic cells, recombinant virus-based methods are mainly used, but since the transduction is performed by centrifugal force, cell stress due to centrifugal force may occur, and thus there is a problem of lowering the viability of transduced cells.

To solve this problem, the use of polycation, spinoculation, recombinant fibronectin fragment coating (e.g., RetroNectin), or biological adjuvant has been attempted, and potential materials for improving transduction have been identified through high-throughput screening. However, in the transduction method using such a method, a large amount of vectors is required for a sufficient level of gene transfer due to a minimum volume required in a standard system to cause problems such as increased costs during the cell transduction process.

Meanwhile, a microfluidic chip refers to a chip that may move a fluid (e.g., a liquid sample) through a microfluidic channel and then be mixed and react with cells in a plurality of chambers in the microfluidic chip. The microfluidic chip is used to significantly reduce the used amount of expensive reagents used for protein and DNA analysis compared to existing methods and reduce the used amount of protein samples or cell samples. Thus, the microfluidic chip has been used to increase accuracy, efficiency, and reliability as well as to create cost and time-reducing effects in fields such as pharmaceuticals, biotechnology, medicine, etc. However, little research has been conducted to apply the microfluidic chip to cell transduction.

Accordingly, the present inventors conducted research to develop an efficient cell transduction method based on a microfluidic chip and completed the present disclosure.

### DISCLOSURE

### Technical Problem

An object of the present disclosure is to provide a cell transduction method including preparing a hydrogel containing a plurality of encapsulated target cells; and contacting viral particles with the target cells in the hydrogel.

Another object of the present disclosure is to provide a microfluidic chip for cell transduction including the following: a virus chamber containing virus particles; a target cell chamber containing target cells; a hydrogel chamber containing a hydrogel before gelation; and a hydrogel microfluidic channel forming portion connected to the virus chamber, the target cell chamber, and the hydrogel chamber through inlets, respectively.

Yet another object of the present disclosure is to provide a microfluidic chip for cell transduction including the following: a virus chamber containing virus particles; and a hydrogel microfluidic channel connected to the virus chamber through an inlet and containing a plurality of encapsulated target cells.

An aspect of the present disclosure provides a cell transduction method including preparing a hydrogel containing a plurality of encapsulated target cells; and contacting viral particles with the target cells in the hydrogel.

### Technical Solution

According to one embodiment of the present disclosure, the target cells may be single-encapsulated.

According to one embodiment of the present disclosure, the hydrogel may be mixed with a culture medium of the target cells.

According to one embodiment of the present disclosure, the contacting may be performed while the virus particles pass through the hydrogel.

According to one embodiment of the present disclosure, the passing may be performed by microfluid flow.

According to one embodiment of the present disclosure, the target cells may be immobilized within the hydrogel.

According to one embodiment of the present disclosure, the pore size of the hydrogel may be 10 nm to 20 μm.

According to one embodiment of the present disclosure, the virus may be any one virus selected from the group consisting of retrovirus, adenovirus, adeno-associated virus, lentivirus, herpes virus and Poxvirus.

According to one embodiment of the present disclosure, the target cells may be immune cells.

Another aspect of the present disclosure provides a microfluidic chip for cell transduction including the following: a virus chamber containing virus particles; a target cell chamber containing target cells; a hydrogel chamber containing a hydrogel before gelation; and a hydrogel microfluidic channel forming portion connected to the virus chamber, the target cell chamber, and the hydrogel chamber through inlets, respectively.

According to one embodiment of the present disclosure, the target cells may be immobilized to the hydrogel microfluidic channel.

According to one embodiment of the present disclosure, the pore size of the hydrogel may be 10 nm to 20 μm.

According to one embodiment of the present disclosure, the virus may be any one virus selected from the group consisting of retrovirus, adenovirus, adeno-associated virus, lentivirus, herpes virus and Poxvirus.

According to one embodiment of the present disclosure, the target cells may be immune cells.

Yet another aspect of the present disclosure provides a microfluidic chip for cell transduction including the following: a virus chamber containing virus particles; and a hydrogel microfluidic channel connected to the virus chamber through an inlet and containing a plurality of encapsulated target cells.

According to one embodiment of the present disclosure, the target cells may be immobilized to the hydrogel microfluidic channel.

According to one embodiment of the present disclosure, the pore size of the hydrogel may be 10 nm to 20 μm.

According to one embodiment of the present disclosure, the virus may be any one virus selected from the group consisting of retrovirus, adenovirus, adeno-associated virus, lentivirus, herpes virus and Poxvirus.

According to one embodiment of the present disclosure, the target cells may be immune cells.

### Advantageous Effects

According to the cell transduction method and the microfluidic chip for cell transduction of the present disclosure, since traits are introduced using a microfluidic-based method, it is possible to avoid cell stress induced by strong centrifugal force and achieve a high multiplicity of infection (MOI) even with a small volume, thereby being efficiently used for cell transformation.

### DESCRIPTION OF DRAWINGS

FIG. 1 is a schematic diagram comparing and illustrating compositions and effects of a conventional cell transduction method (standard system) and a microfluidic transduction method (microfluidic transduction platform).

FIG. 2 is a photograph showing comparing cell shapes according to a hydrogel mixing ratio.

FIG. 3 is a graph showing a change in cell number according to a hydrogel mixing ratio.

FIG. 4 is a graph showing a change in cell viability according to a hydrogel mixing ratio.

FIG. 5 is a diagram illustrating a configuration of a microfluidic chip according to an embodiment of the present disclosure.

FIG. 6 is a photograph showing results of viral transduction using a microfluidic chip according to an embodiment of the present disclosure.

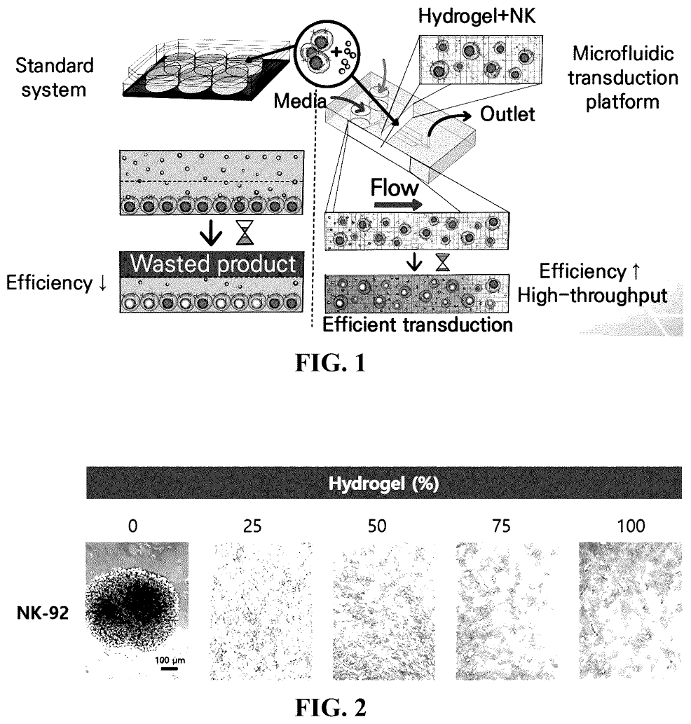

### BEST MODE FOR CARRYING OUT THE INVENTION

An aspect of the present disclosure provides a cell transduction method including preparing a hydrogel containing a plurality of encapsulated target cells; and contacting viral particles with the target cells in the hydrogel.

The present disclosure relates to a microfluidic method for improving cell stress and low transduction rate caused by centrifugal force occurring in conventional cell transduction methods. In the present disclosure, since traits are introduced by displacing target cells to be transduced in a microfluidic channel and contacting viral particles containing the introduced traits with the target cells through microfluid flow, unlike a conventional centrifugal force method, there is no cell stress caused by centrifugal force, so that cell viability is excellent. In addition, according to a conventional method of introducing traits by inserting viral particles containing the introduced traits into a culture medium of the target cells immobilized to the bottom, there was a problem in increased cost during the transduction process due to low transduction efficiency. However, according to the microfluidic method according to the present disclosure, the interaction between the target cells and the virus particles is increased by flowing the virus particles, and thus, it is possible to achieve a high multiplicity of infection (MOI) even with a small volume of virus particles.

The cell transduction method of the present disclosure may be performed by the following method.

The first step of the present disclosure is a step of preparing a hydrogel containing a plurality of encapsulated target cells.

As used herein, the term “hydrogel” refers to a hydrophilic polymer crosslinked by cohesive forces such as covalent bonds, hydrogen bonds, van der Waals bonds, or physical bonds, and includes a material with a 3D polymer network structure that may swell in an aqueous solution by containing a large amount of water.

The hydrogel that may be used herein may be a hydrogel capable of culturing cells, and may be prepared with one or more materials selected from the group consisting of, for example, various synthetic materials such as poly(ethylene oxide) (PEO), poly(vinyl alcohol) (PVA), poly(acrylic acid) (PAA), and natural materials such as agarose, alginate, chitosan, collagen, fibrin, gelatin, and hyaluronic acid, preferably matrigel, but is not limited thereto.

According to one embodiment of the present disclosure, the hydrogel may be mixed with a culture medium of the target cells.

The hydrogel of the present disclosure may be mixed and used with the medium of the cells to be transduced, and in this case, the hydrogel is preferably mixed at a ratio of 50% or more.

Gelation of the hydrogel may be achieved by chemically or physically crosslinking polymer chains, caused by chemical agents (e.g., cross-linking agents) or physical stimuli (e.g., pH and/or temperature) to form a network structure. In the gelation process to contain target cells, 2D and 3D structures may be formed using hydrogel patterning technology, and one or more patterns may be integrated by changing the length, width, and height.

According to one embodiment of the present disclosure, the target cells may be immobilized within the hydrogel.

The hydrogel prepared in this step may be formed by encapsulating a plurality of target cells at regular intervals within the 3D structure of the hydrogel. For example, for target cell encapsulation in the hydrogel, a method of X. Wang, J. A. et al., Biomaterials. 29 (2008) 10541064 may be used, but is not limited thereto, and in the related art, any method of encapsulating and preparing cells in a hydrogel may be used without limitation.

For example, in the case of a matrigel, which gelates by a temperature, cells may be mixed with the matrigel and left at 37° C. to be encapsulated and gelate, and in the case of collagen, which gelates by pH, a collagen solution may be neutralized to about pH 7.4, mixed with cells, and left at 37° C. to be encapsulated and gelate. In the case of sodium alginate, which gelates by Ca²⁺ ions, the cells may be mixed with a sodium alginate solution and immersed in a CaCl₂) solution to be encapsulated and gelate, and in the case of gelatin methacrylate (GelMA), which gelates by UV, cells may be mixed in a solution containing GelMA and a photoinitiator such as iragacure 2959 to be encapsulated and gelate using UV.

According to one embodiment of the present disclosure, the target cells may be single-encapsulated.

The mechanical and biological properties of a hydrogel containing a plurality of single-encapsulated target cells may be adjusted depending on transduction conditions of the target cells, such as the type of cells to be used and the size of virus particles to be used.

The cells used in the first step may be stem cells or progenitor cells. For example, the stem cells or progenitor cells may be embryonic stem cells or induced pluripotent stem cells, and the stem cells or progenitor cells may also be mesenchymal stem cells, hematopoietic stem cells, neural stem cells, retinal stem cells, myocardial stem cells, skeletal muscle stem cells, adipose tissue-derived stem cells, chondrogenic stem cells, liver stem cells, kidney stem cells, or pancreatic stem cells, and may be hematopoietic stem cells or hematopoietic progenitor cells, but are not limited thereto.

In addition, the cells used in the step may be osteoblasts, chondrocytes, adipocytes, skeletal muscles, myocardium, nerves, astrocytes, oligodendrocytes, Schwann cells, retina cells, corneal cells, skin cells, monocytes, macrophages, neutrophils, basophils, eosinophils, erythrocytes, megakaryocytes, dendritic cells, T-lymphocytes, B-lymphocytes, NK-cells, gastric cells, intestinal cells, smooth muscle cells, vascular cells, bladder cells, pancreatic alpha cells, pancreatic beta cells, pancreatic delta cells, liver cells, kidney cells, adrenal cells, or lung cells, but are not limited thereto.

The second step of the present disclosure is a step of contacting the viral particles with the target cells in the hydrogel.

According to one embodiment of the present disclosure, the virus may be any one virus selected from the group consisting of retrovirus, adenovirus, adeno-associated virus, lentivirus, herpes virus and Poxvirus.

As used herein, the viral particles may be viral vectors containing genetic traits to be introduced into target cells, and may be, for example, adenovirus vector, retrovirus vector, lentivirus vector, adeno-associated virus (AAV) vector, herpes virus vector, or Poxvirus vector, but are not limited thereto.

According to one embodiment of the present disclosure, the pore size of the hydrogel may be 10 nm to 20 μm.

The pore size of the hydrogel may be 10 nm to 50 μm, but preferably 10 nm to 20 μm.

According to one embodiment of the present disclosure, the contacting may be performed while the virus particles pass through the hydrogel.

According to one embodiment of the present disclosure, the passing may be performed by microfluid flow.

In the present disclosure, the transduction may be performed by disposing target cells in a microfluidic channel and contacting the target cells with viral particles containing genetic traits to be introduced into the target cells through microfluid flow.

Depending on the pore size of the hydrogel, the flow of the virus particles and the flow time may be determined. That is, the pore size of the hydrogel may be determined considering a type of viral vector to be used, the size of a genetic trait to be introduced, and a type of target cell. In addition, since virus particles larger than the pore size of the hydrogel are difficult to flow, transduction into target cells may be prevented for virus particles introduced with incorrect traits that are larger than the introduced traits.

According to one embodiment of the present disclosure, the target cells may be immune cells.

Due to the nature of immune cells, since there is a possibility of inducing apoptosis during transduction and the efficiency is low, a high MOI is required. In particular, in the case of primary NK cells, since the expression of a VSV-G receptor is low, there is a problem that the transduction efficiency of general lentivirus is low. On the other hand, according to the transduction method according to the present disclosure, since it is possible to achieve a high MOI with a small volume of virus particles without using centrifugal force, the transduction method may be effectively applied to virus-based immune cell transduction, for example, lentivirus-based NK cell transduction.

Another aspect of the present disclosure provides a microfluidic chip for cell transduction including the following: a virus chamber containing virus particles; a target cell chamber containing target cells; a hydrogel chamber containing a hydrogel before gelation; and a hydrogel microfluidic channel forming portion connected to the virus chamber, the target cell chamber, and the hydrogel chamber through inlets, respectively.

Immediately before introducing the virus particles into the target cells, mixing and gelation of the hydrogel before gelation with the target cells are performed in the hydrogel microfluidic channel forming portion of the microfluidic chip of the present disclosure to form a hydrogel microfluid channel containing the target cells. After the hydrogel microfluidic channel containing the target cells is formed, the virus particles are injected into the hydrogel microfluidic channel and flow along the hydrogel microfluidic channel. In this process, the traits contained in the virus particles are introduced into the target cells by contacting the virus particles with the target cells that are encapsulated in the hydrogel and distributed at regular intervals.

The virus particles may flow alone in the hydrogel microfluidic channel or may move together during the flow of the culture medium of the target cells, and in this case, the virus chamber may further include the culture medium of the target cells.

According to one embodiment of the present disclosure, the hydrogel may be mixed with the culture medium of the target cells.

The target cell culture medium of the present disclosure may be used without limitation as long as the culture medium is a medium suitable for culturing the target cells included in the hydrogel.

The virus particles may further include a buffer and the like to maintain the activity of the virus particles.

According to one embodiment of the present disclosure, the target cells may be immobilized to the hydrogel microfluidic channel.

According to one embodiment of the present disclosure, the pore size of the hydrogel may be 10 nm to 20 μm.

According to one embodiment of the present disclosure, the virus may be any one virus selected from the group consisting of retrovirus, adenovirus, adeno-associated virus, lentivirus, herpes virus and Poxvirus.

According to one embodiment of the present disclosure, the target cells may be immune cells.

Yet another aspect of the present disclosure provides a microfluidic chip for cell transduction including the following: a virus chamber containing virus particles; and a hydrogel microfluidic channel connected to the virus chamber through an inlet and containing a plurality of encapsulated target cells.

The microfluidic chip of the present disclosure may have a hydrogel microfluidic channel containing target cells already formed on the microfluidic chip. In this case, the hydrogel microfluidic channel may be formed when a mixture of the hydrogel before gelation and the target cells gelates on the microfluidic chip.

According to one embodiment of the present disclosure, the target cells may be immobilized to the hydrogel microfluidic channel.

According to one embodiment of the present disclosure, the pore size of the hydrogel may be 10 nm to 20 μm.

According to one embodiment of the present disclosure, the virus may be any one virus selected from the group consisting of retrovirus, adenovirus, adeno-associated virus, lentivirus, herpes virus and Poxvirus.

According to one embodiment of the present disclosure, the target cells may be immune cells.

### MODE FOR CARRYING OUT THE INVENTION

Hereinafter, the present disclosure will be described in more detail with reference to one or more Examples. However, these Examples are only illustrative of the present disclosure, and the scope of the present disclosure is not limited to these Examples.

### Example 1. Change in Cell Viability According to Encapsulation Using Hydrogel

Cell viability according to encapsulation using a hydrogel of the present disclosure was confirmed.

Specifically, the cells were prepared by using matrigel as the hydrogel and mixing a cell medium at volume ratios of 0, 25, 50, 75, and 100%, respectively, added in the same number of an NK-92 cell hydrogel, respectively, and then left at 37° C., encapsulated and gelate, and then cultured in a CO₂ incubator for 2 days, and thereafter, the cells were observed under a microscope. In addition, the cell number and cell viability of NK-92 cells were confirmed by staining with trypan blue at a ratio of 1:1 and counting the number of unstained living cells.

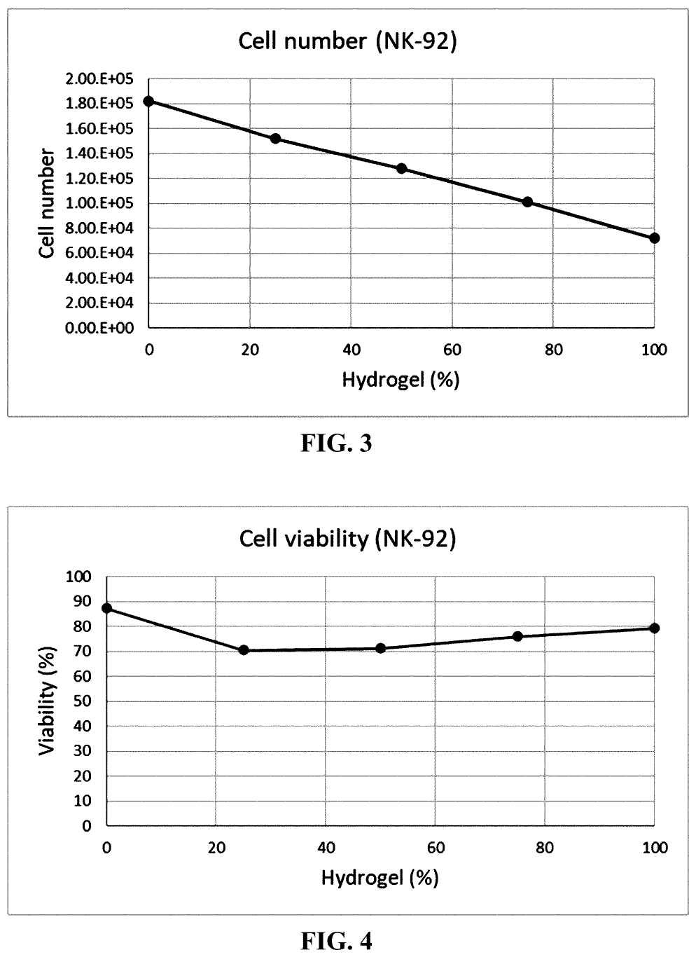

As a result, it was confirmed that a gel state suitable for cell encapsulation was made at a ratio of 50% or more with the medium (FIG. 2). In addition, it was shown that the total number of living cells decreased as the ratio of hydrogel increased compared to a control group without mixing a hydrogel, but it was confirmed that the cell viability remained 70% or more regardless of the mixing ratio of the hydrogel (FIGS. 3 and 4).

Through these results, it was confirmed that when encapsulating the cells by mixing 50% of the hydrogel and injecting the encapsulated cells into the chip, it was possible to maintain the gel state and maintain the viability of the cells capable of performing a transduction experiment.

### Example 2. Effect of Improving Viral Transduction Using Microfluidic Chip

Transduction efficiency was compared to determine whether viral transduction was improved using a microfluidic chip.

Specifically, the transduction efficiency was analyzed by preparing lentivirus capable of expressing green fluorescent protein (GFP) in the cytoplasm of the cell, and using NK-92 cultured in a MEM alpha medium containing 0.2 mM myo-inositol, 0.1 mM 2-mercarptoethanol, 0.02 mM folic acid, 12.5% horse serum, 12.5% FBS and 100 IU/mL IL-2. The medium was purchased from Thermo Fisher Scientific (Waltham, USA), and the materials to be added were purchased and used from Sigma Aldrich (St. Louis, USA) and BD (Franklin Lakes, USA).

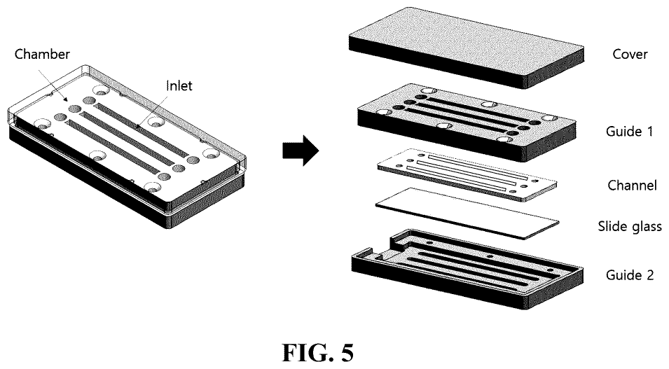

As shown in FIG. 5, a chip consisting of a cover, a guide, a PDMS chip, and a slide glass was manufactured, and matrigel and each medium were mixed at 5:5 through an inlet and injected together with the cells, and gelation was performed for 10 minutes. Then, virus and 8 μg/mL of polybrene were mixed, added to each side chamber by 50 μL each, and placed on a rocker moving at a speed of 10 rpm at an angle of 7° in a CO₂ incubator. After 12 hours of incubation, the virus was removed, and a new medium was injected and cultured for 3 days, and then the results were observed. In addition, for comparison, a control group without virus and virus and 8 μg/mL of polybrene were added together with the cells, and experimented together with a spinoculation method of infection using centrifugal force at a speed of 1200×g for 30 minutes, and after 12 hours, the virus was removed in the same manner as described above, and then a new medium was injected and cultured for 3 days.

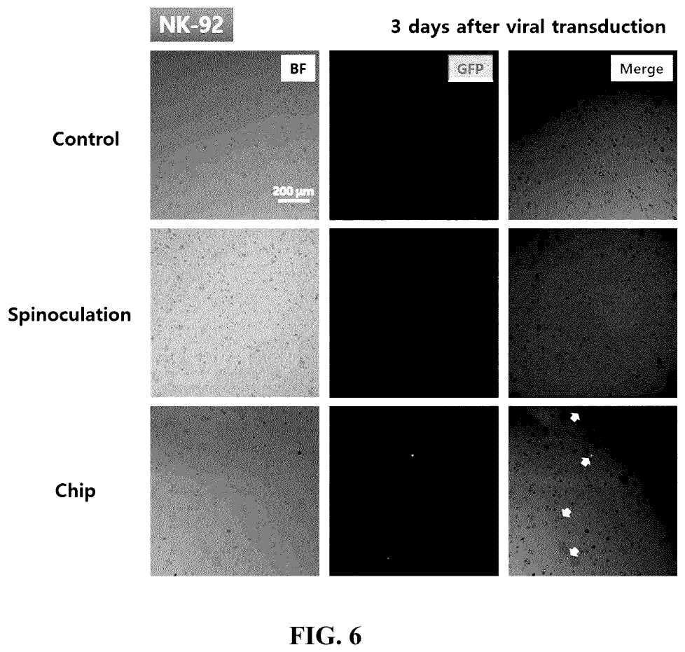

As a result, unlike the existing method, in the experiment using the microfluidic chip, it was showed that virus transduction was successful and cells expressing GFP were confirmed (FIG. 6, yellow arrow).

Through these results, it was confirmed that by using the microfluidic chip according to one embodiment of the present disclosure, it is possible to effectively transduce immune cells such as NK-92, which had low viral transduction efficiency due to existing experimental methods.

The present disclosure has been described above with reference to preferred embodiments thereof. It will be understood to those skilled in the art that the present disclosure may be implemented as a modified form without departing from an essential characteristic of the present disclosure. Therefore, the disclosed embodiments should be considered in an illustrative viewpoint rather than a restrictive viewpoint. The scope of the present disclosure is defined by the appended claims rather than by the foregoing description, and all differences within the scope of equivalents thereof should be construed as being included in the present disclosure.

1. A cell transduction method, comprising:
preparing a hydrogel containing a plurality of encapsulated target cells; and
contacting viral particles with the target cells in the hydrogel.

2. The cell transduction method of claim 1, wherein the target cells are single-encapsulated.

3. The cell transduction method of claim 1, wherein the hydrogel is mixed with a culture medium of the target cells.

4. The cell transduction method of claim 1, wherein the contacting is performed while the virus particles pass through the hydrogel.

5. The cell transduction method of claim 4, wherein the passing is performed by microfluid flow.

6. The cell transduction method of claim 1, wherein the target cells are immobilized within the hydrogel.

7. The cell transduction method of claim 1, wherein a pore size of the hydrogel is 10 nm to 20 μm.

8. The cell transduction method of claim 1, wherein the virus is any one virus selected from the group consisting of retrovirus, adenovirus, adeno-associated virus, lentivirus, herpes virus and Poxvirus.

9. The cell transduction method of claim 1, wherein the target cells are immune cells.

10. A microfluidic chip for cell transduction, comprising the following:
a virus chamber containing virus particles;
a target cell chamber containing target cells;
a hydrogel chamber containing a hydrogel before gelation; and
a hydrogel microfluidic channel forming portion connected to the virus chamber, the target cell chamber, and the hydrogel chamber through inlets, respectively.

11. The microfluidic chip of claim 10, wherein the target cells are immobilized to the hydrogel microfluidic channel.

12. The microfluidic chip of claim 10, wherein a pore size of the hydrogel is 10 nm to 20 μm.

13. The microfluidic chip of claim 10, wherein the virus is any one virus selected from the group consisting of retrovirus, adenovirus, adeno-associated virus, lentivirus, herpes virus and Poxvirus.

14. The microfluidic chip of claim 10, wherein the target cells are immune cells.

15. A microfluidic chip for cell transduction, comprising the following:
a virus chamber containing virus particles; and
a hydrogel microfluidic channel connected to the virus chamber through an inlet and containing a plurality of encapsulated target cells.

16. The microfluidic chip of claim 15, wherein the target cells are immobilized to the hydrogel microfluidic channel.

17. The microfluidic chip of claim 15, wherein the pore size of the hydrogel is 10 nm to 20 μm.

18. The microfluidic chip of claim 15, wherein the virus is any one virus selected from the group consisting of retrovirus, adenovirus, adeno-associated virus, lentivirus, herpes virus and Poxvirus.

19. The microfluidic chip of claim 15, wherein the target cells are immune cells.
